(19) United States
(12) Patent Application Publication
(10) Pub. No.: US 2005/0006171 A1
(43) Pub. Date: Jan. 13, 2005

(54) Adaptor for mounting a sound tube in an earpiece

(75) Inventor: Klaus Sommer Ipsen, Vanlose (DK)

Correspondence Address: David G. Beck;Bingham McCutchen LLP, 18th Floor, Three Embarcadero Center, San Francisco, CA, 94111, US

(73) Assignee: GN ReSound A/S, Taastrup (DK)

(21) Appl. No.: 10/778,716

(22) Filed: Feb. 13, 2004

(30) Foreign Application Priority Data
Feb. 14, 2003 (DK) PA 2003 00230

(51) Int. Cl. A61B 7/02

(52) U.S. Cl. 181/135

(57) ABSTRACT

The present invention relates to mounting of a sound tube inside a shell of a hearing aid. The invention provides a sound tube adaptor, a tool for insertion of the adaptor and a method for attaching a sound tube to a shell, the sound tube adaptor facilitating attachment of a sound tube to a shell, having a first end for attachment to one end of a sound tube and a second end that is adapted to be received inside the shell in a hole in the shell in such a way that the adaptor and the sound tube extend inside the shell from the hole towards the entrance of an ear canal of a user when the shell is positioned in the ear canal.

### FIELD OF THE INVENTION

The present invention relates to mounting of a sound tube inside a shell of a hearing aid.

### PRIOR ART

Behind-the-ear hearing aids in which a sound tube conducts sound generated by the receiver of the hearing aid into the ear canal are well known in the art. In order to position the sound tube securely and comfortably in the ear canal, a shell is provided for insertion into the ear canal of the user. Typically, the shell is individually adapted to the human anatomy of the ear of the user.

So-called “open” shells are generally preferred in order to affect the ear canal as little as possible by avoiding blockage of the ear canal, i.e. the occlusion effect. This also assists in maintaining the natural hearing capacity of the user.

### SUMMARY OF THE INVENTION

It is desirable to provide a shell that facilitates attachment of the sound tube to the shell and that also has a vent for at least partially reducing the occlusion effect. Further, the vent assists in maintaining the natural hearing capacity of the user.

According to a first aspect of the invention, a sound tube adaptor is provided that may be attached to a sound tube at one end and inserted into a hole in a shell at the other end. After insertion, the adaptor and the sound tube extend inside the shell from the hole towards the entrance of an ear canal of a user when the shell is positioned in the ear canal.

After insertion, the adaptor and the hole may co-operate to provide at least one vent.

According to a second aspect of the invention, a shell is provided having a hole for receiving and holding a sound tube adaptor with a sound tube. The adaptor and the hole may co-operate to provide at least one vent when the adaptor is inserted in the shell hole.

According to a third aspect of the invention, a method of attaching a sound tube to a shell is provided comprising the steps of attaching one end of the sound tube to the above-mentioned adaptor, inserting the other end of the sound tube into a hole in the shell from the outside of the shell, passing the sound tube through the hole, and mounting the adaptor in the hole whereby the sound tube is attached to the shell.

According to a fourth aspect of the invention, a tool is provided for insertion of the adaptor into the hole of the shell.

The tool may have a first protrusion that is adapted for insertion into an output opening of the adaptor, and a second protrusion wherein the space between the first and second protrusions is adapted to accommodate a wall of the adaptor.

The tool may further have a center hole for checking that the adaptor may be used with a particular shell, the center hole having a diameter so that the cross-section of the end of the particular shell with the hole can cover the center hole when the shell has a sufficient size for accommodating an adaptor.

Still further an end of the tool may be dimensioned so that the end can enter the shell hole when the shell has a sufficient size for accommodation of an adaptor.

The sound tube adaptor may have a first end of a generally cylindrical shape extending along a longitudinal axis. Preferably, a bead is provided at the first end for insertion into the sound tube for secure attachment by resilience of the sound tube.

The sound tube adaptor may have a second end for attachment to the shell with a generally cylindrical shape, or a generally tapered cylindrical shape with a cross-section that increases or decreases along the longitudinal axis of the adaptor. In one embodiment, the cross-section of the adaptor at the second end has a cross-section that is less than the corresponding cross-section of the shell hole, so that an area of the hole, after insertion of the adaptor, may function as at least one vent. The at least one vent may constitute a single opening in the shell with the attached adaptor, or, the at least one vent may constitute a plurality of separate openings in the shell with the attached adaptor.

Further, the sound tube adaptor may have at least one first flange at the second end for abutment with the outer surface of the shell after insertion of the adaptor in the shell hole.

At least one second flange may be positioned at a distance from the at least one first flange along the longitudinal axis. The distance may be approximately equal to the thickness of the shell hole so that the at least first flange abuts the outer surface of the shell and the at least second flange abuts the inner surface of the shell after insertion of the adaptor in the shell hole.

Further, the at least second flange may be tapered along the longitudinal axis towards the first end whereby the adaptor may be fixed in the shell hole by a snap fit coupling.

In a preferred embodiment, the at least second flange is adapted to engage with protrusions provided in the shell at the hole for prevention of rotational movement of the adaptor in the hole.

The sound tube adaptor may further comprise an end plate at the second end having a vent hole.

In a preferred embodiment, a sound tube adaptor further contains weakenings at the second end allowing removal of part of the adaptor for obstruction of a smaller part of the hole in the shell after insertion of the adaptor.

Preferably, the hole and the sound adaptor are designed so that the sound adaptor fits in the hole in two different positions mutually angularly separated 180°. This adds flexibility to the system in that the shell may not be able to accommodate the adaptor and the sound tube in one of the positions, however in embodiments with an additional second position, this position may then be used.

In still another embodiment, the adaptor and the hole are designed so that the adaptor may be fitted into the hole in any arbitrary angular position further facilitating accommodation of the sound tube and the adaptor in the shell.

The shell may further comprise an end plate containing the hole. According to this embodiment, an appropriate shell may be individually produced for the user or, an appropriate standard size may be selected, and the dispenser may select an end plate with an appropriately sized and shaped hole. The end plate is fitted to the shell geometry and fixed to the shell in a way well known in the art of assembling CIC- and ITE-shells with a faceplate.

The adaptor and the sound tube may be integrated into one component for ease of manufacture and assembly.

In an embodiment with a separate adaptor and sound tube, individual adjustment of the distance from the adaptor to a first bend of the sound tube is facilitated. The sound tube may be cut at the desired length before attachment to the adaptor.

Although a shell for a behind-the-ear hearing aid has been mentioned above, it should be understood that the invention covers any shells requiring mounting of a sound tube, such as shells for completely-in-the-canal (CIC) hearing aids, for in-the-canal (ITE) hearing aids, for behind-the-ear (BTE) hearing aids, for earplugs for protection against noise with two-way communication means, for audio equipment, etc.

### BRIEF DESCRIPTION OF THE DRAWINGS

Below, the invention will be further described and illustrated with reference to the accompanying drawings in which:

FIG. 1 is a perspective view of a first embodiment of the invention,

FIG. 2 is a perspective view of a second embodiment of the invention,

FIG. 3 is a perspective view of an end-plate according to the invention,

FIG. 4 shows attachment according to the invention of an adaptor to an end-plate and a sound tube,

FIG. 5 is a perspective view of a third embodiment of the invention,

FIG. 6A is a perspective view of a fourth embodiment of the invention,

FIG. 6B is a perspective view of a fifth embodiment of the invention,

FIG. 7 is a perspective view of a sixth embodiment of the invention,

FIG. 8 is a perspective view of an open shell without an end-plate,

FIG. 9 is a perspective view of the open shell with the adaptor and sound tube,

FIG. 10 is another perspective view of the open shell with the adaptor and sound tube,

FIG. 11 is yet another perspective view of the open shell with the adaptor and sound tube,

FIG. 12 is a perspective view of a tool for inserting the adaptor into the hole of the shell,

FIG. 13 illustrates operation of the tool for insertion of the adaptor, and

FIG. 14 illustrates the position of the tool immediately before removal of the tool.

### DETAILED DESCRIPTION OF PREFERRED EMBODIMENTS

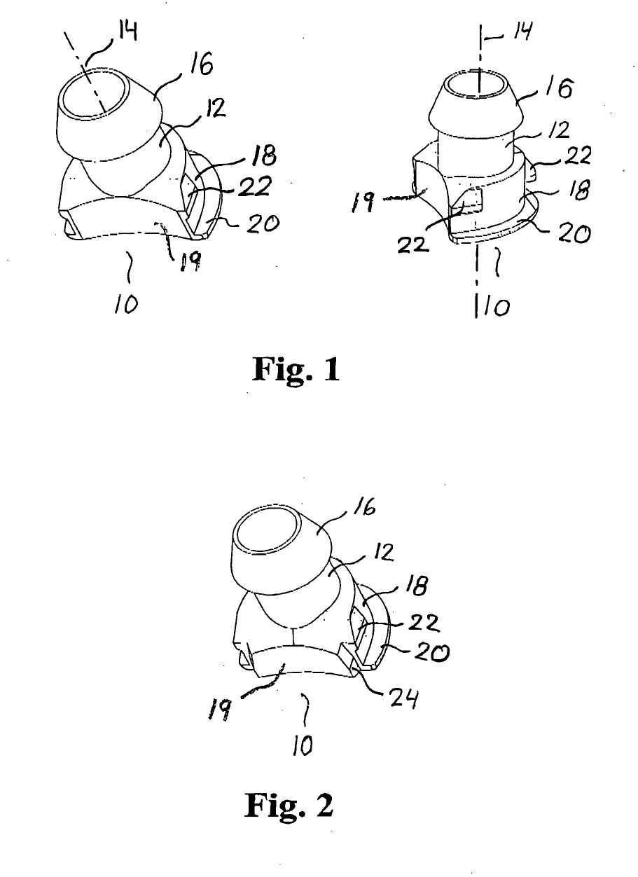

FIG. 1 shows a sound tube adaptor 10 according to the present invention. The sound tube adaptor 10 has a first end 12 of a generally cylindrical shape extending along a longitudinal axis 14. A bead 16 is provided at the first end 12 for insertion into the sound tube for secure attachment by resilience of the sound tube.

The sound tube adaptor 10 has a second end 18 for attachment to the shell with a generally cylindrical shape. A first flange 20 is provided at the second end 18 for abutment with the outer surface of the shell after insertion of the adaptor 10 in the shell hole. Further, at least one second flange 22 is positioned at a distance from the at least one first flange 20 along the longitudinal axis 14. The distance is approximately equal to the thickness of the shell hole so that the first flange 20 abuts the outer surface of the shell and each of the at least second flanges 22 abuts the inner surface of the shell after insertion of the adaptor 10 in the shell hole.

FIG. 2 shows another sound tube adaptor 10 according to the present invention, with a protrusion 24 extending from the main body of the adaptor 10 whereby the cross-section of the un-obstructed part of the hole in the shell providing a vent is less than the corresponding cross-section for the embodiment shown in FIG. 1.

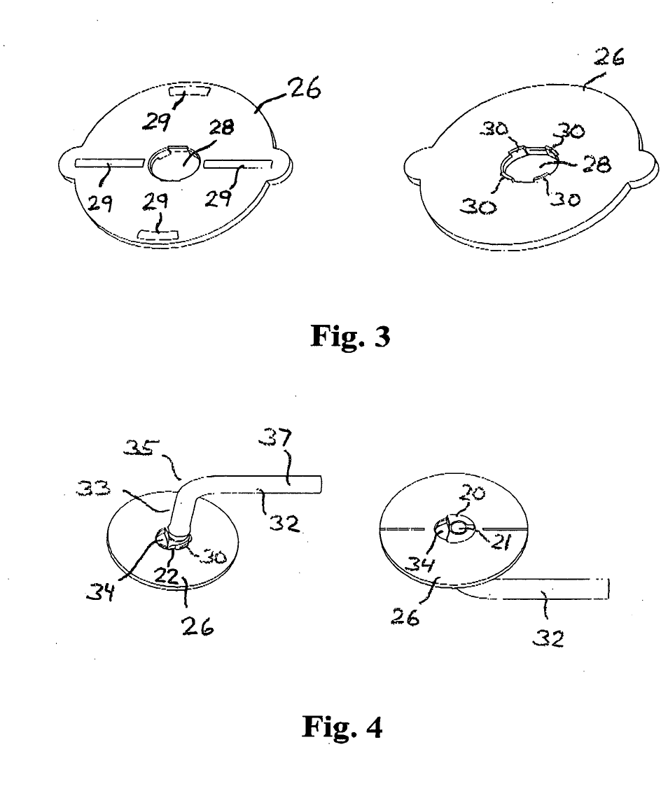

FIG. 3 shows an end plate 26 containing the hole 28. Protrusions 30 are provided at the hole 28 for engagement with the at least second flange 22 while the at least first flange 20 engages with the side of the end plate 26 opposite the protrusions 30 whereby rotational movement of the adaptor 10 in the hole 28 is prevented. The end-plate 26 also has raised portions 29 facilitating correct positioning of the adaptor in relation to the hole 28 and for supporting the end-plate resting on a surface and accommodating the adaptor 10.

FIG. 4 illustrates assembly of the sound tube 32 with the sound tube adaptor 10 and the end plate 26. The sound tube 32 has a first straight portion 33, a first bend 35, and a second straight portion 37. When the shell is positioned in the user's ear, the first portion 33 is substantially horizontal and the second portion 37 is substantially vertical.

It should be noted that the cross-section of the adaptor 10 at its second end 18 has a cross-section that is less than the corresponding cross-section of the end-plate hole 28, so that an area 34 of the hole, after insertion of the adaptor 10, functions as a vent 34.

As further illustrated in FIG. 4, the at least second flange 22 is adapted to engage with the protrusions 30 provided at the hole 28 for prevention of rotational movement of the adaptor 10 in the hole 28.

The first end 12 of the adaptor 10 with its bead 16 is inserted into the sound tube 32 for secure attachment by resilience of the sound tube 32.

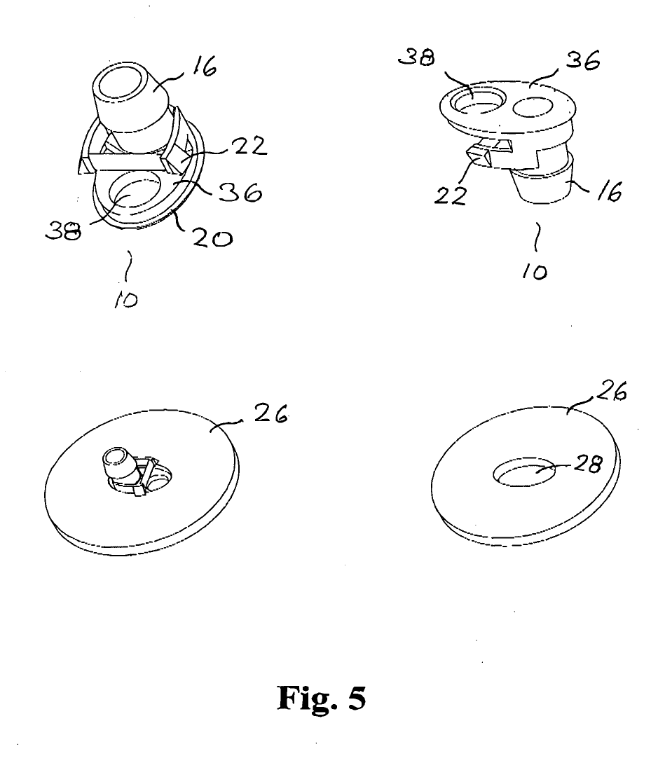

FIG. 5 illustrates yet another embodiment of the invention, wherein the sound tube adaptor 10 further comprises an end-plate 36 at the second end 18 having a vent hole 38. In the shown embodiment, the end plate 36 has a generally oval shape fitting the oval shape of the end-plate hole 28. It should be noted that after insertion of the adaptor 10 in the end-plate hole 28, the at least one first flange 20 and the at least one second flange 22 engage with respective opposite sides of the end plate 26 for secure attachment of the adaptor 10 in the hole 28.

It should also be noted that the at least second flange 22 are tapered along the longitudinal axis 14 of the adaptor 10 towards the first end 12 whereby the adaptor 10 is inserted into the end-plate hole 28 by a snap fit coupling.

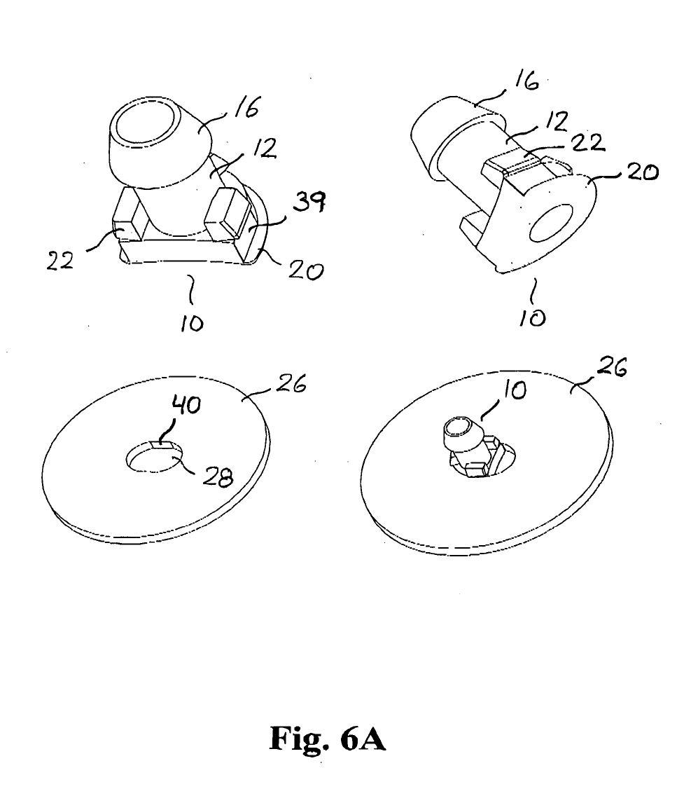

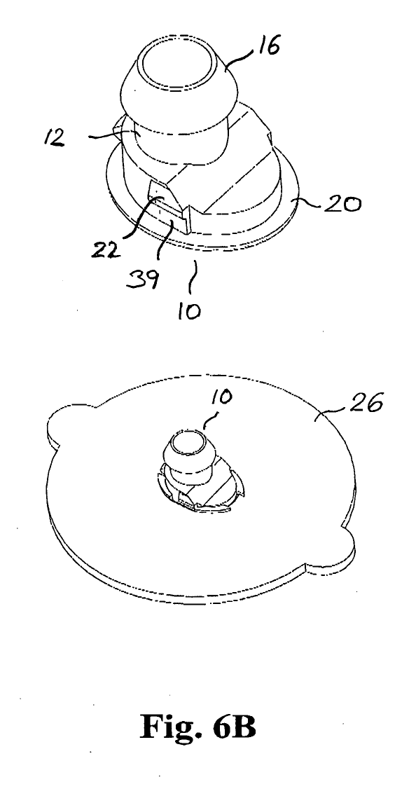

FIG. 6A illustrates still another embodiment of the invention that corresponds to the embodiment shown in FIG. 1, however the generally oval circumference of the adaptor 10 and the hole 28 of the embodiment of FIG. 1 further contains linear segments 39, 40, respectively, for fixed attachment of the adaptor 10 in the hole 28.

FIG. 6B illustrates an embodiment wherein the cross-section of the second end of the adaptor 10 corresponds to the hole 28 in the shell or the shell end-plate in such a way that after insertion of the adaptor 10 in the hole 28 there is no un-obstructed part of the hole 28, i.e. the adaptor 10 according to this embodiment does not cooperate with the hole 28 to form a vent.

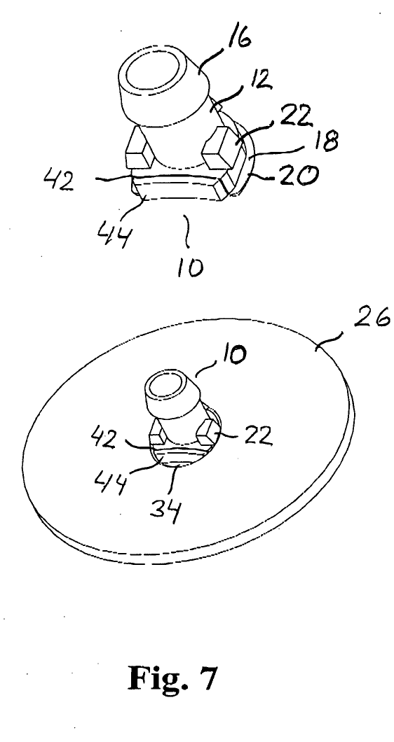

In the embodiment shown in FIG. 7, the sound tube adaptor 10 further contains weakenings 42 at the second end 18 allowing removal of a part 44 of the adaptor 10 for obstruction of a smaller part 34 of the hole 28 in the shell or end plate 26 after insertion of the adaptor 10.

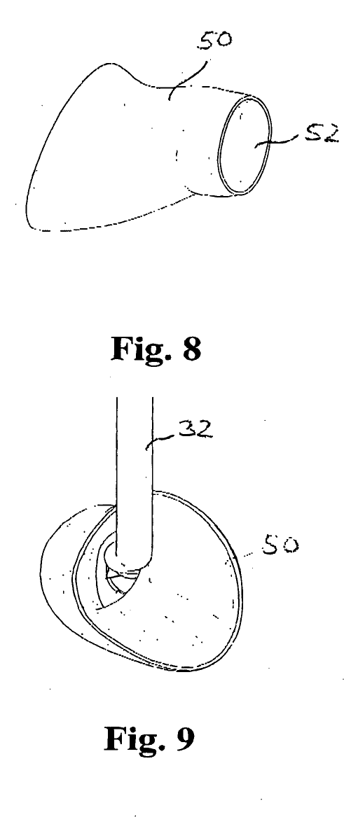

FIG. 8 illustrates a hollow shell 50 with an aperture 52 for reception of the end plate 26.

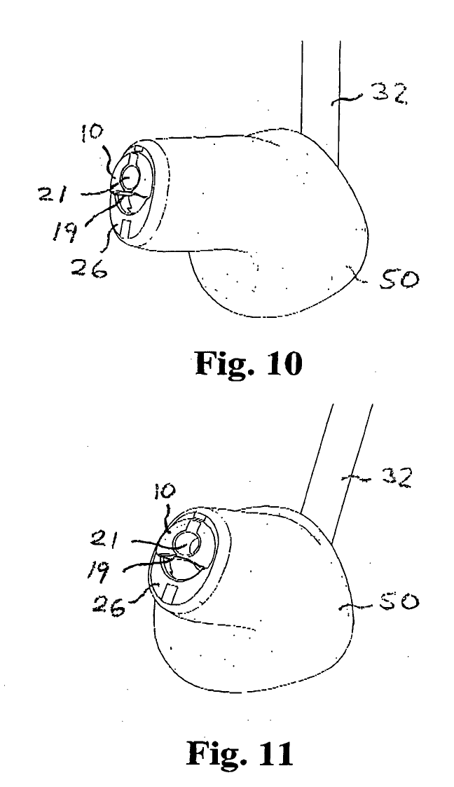

FIGS. 9-11 show various perspective views of the assembled shell ready for mounting in the user's ear.

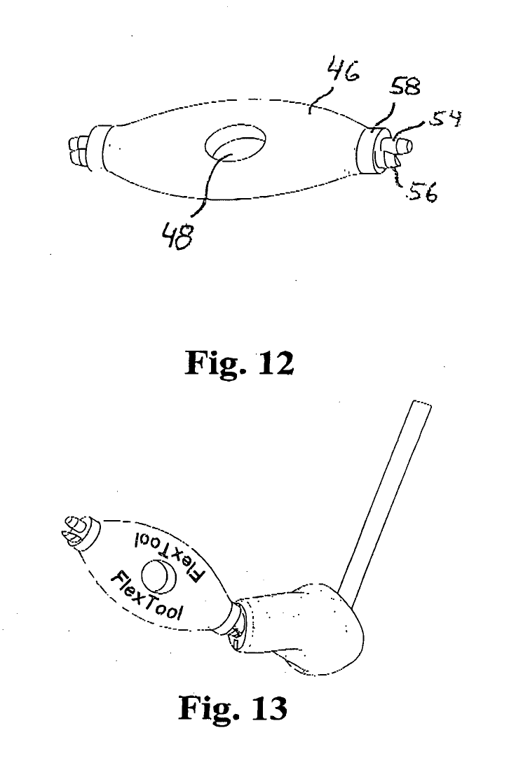

FIG. 12 shows a tool 46 for insertion of the adaptor 10 with a sound tube 32 into the end-plate hole 28. The tool 46 has a center hole 48 for checking that the adaptor assembly may be used for a particular shell 50. If the cross-section of the aperture 52 covers the center hole 48, the shell 50 has a sufficient size for accommodating an adaptor 10. Likewise, if the end 58 of the tool can enter the opening 52 of the shell 50, the shell 50 has a sufficient size for accommodating an adaptor 10. The protrusion 54 is adapted for insertion into output opening 21 of the adaptor 10, and the space between the flat protrusion 56 and the round protrusion 54 is adapted to accommodate the cut side 19 of the adaptor 10.

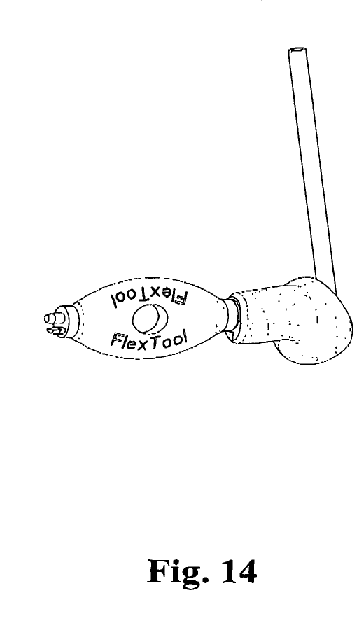

During insertion with the tool 46, the adaptor 10 with the sound tube is removably attached to the tool 46 by insertion of the round protrusion 54 into the adaptor opening 21 and accommodation of the cut side 19 of the adaptor between the protrusions 54, 56. The sound tube 32 is then inserted into the end-plate hole 28 until the tool 46 abuts the end plate 26. As shown in FIG. 13, the tool 46 is angled in relation to the end plate so that the flanges opposite the cut side 19 of the adaptor 10 engage with the edge of the hole 28. Upon engagement, the tool is moved to the position shown in FIG. 14, and the adaptor snaps into its position in the hole by engagement of further flanges. Finally, the tool is removed. The adaptor 10 may be removed from the shell 50 by performing the above-mentioned steps in the opposite order.

1. A sound tube adaptor facilitating attachment of a sound tube to a shell, having a first end for attachment to one end of a sound tube and a second end that is adapted to be received inside the shell in a hole in the shell in such a way that the adaptor and the sound tube extend inside the shell from the hole towards the entrance of an ear canal of a user when the shell is positioned in the ear canal.

2. An adaptor according to claim 1, wherein the adaptor and the hole co-operate to provide at least one vent when the adaptor is inserted in the shell hole.

3. An adaptor according to claim 1, wherein the first end has a generally cylindrical shape extending along a longitudinal axis and a bead at the first end for insertion into the sound tube for secure attachment by resilience of the sound tube.

4. An adaptor according to claim 1, wherein the second end for attachment to the shell has a generally cylindrical shape, and wherein the cross-section of the adaptor at the second end has a cross-section that is less than the corresponding cross-section of the shell hole, so that an area of the hole, after insertion of the adaptor, may function as a vent.

5. An adaptor according to claim 4, having at least one first flange at the second end for abutment with the outer surface of the shell after insertion of the adaptor in the shell hole.

6. An adaptor according to claim 5, further having at least one second flange positioned at a distance from the at least one first flange along the longitudinal axis that is approximately equal to the thickness of the shell hole for abutment with the inner surface of the shell after insertion of the adaptor in the shell hole.

7. An adaptor according to claim 6, wherein the at least second flange is tapered along the longitudinal axis towards the first end whereby the adaptor may be fixed in the shell hole by a snap fit coupling.

8. An adaptor according to claim 5, wherein the at least second flange is adapted to engage with protrusions provided in the shell at the hole for prevention of rotational movement of the adaptor in the hole.

9. An adaptor according to claim 1, further comprising an end plate at the second end having a vent hole.

10. An adaptor according to claim 1, further containing weakenings at the second end allowing removal of part of the adaptor for obstruction of a smaller part of the hole in the shell after insertion of the adaptor.

11. A shell having a hole that is adapted to receive and hold an adaptor with a sound tube.

12. A shell according to claim 11, wherein the adaptor and the hole co-operate to provide a vent when the adaptor is inserted in the shell hole.

13. A shell according to claim 12, wherein the cross-section of the hole is larger than the corresponding cross-section of the adaptor, so that an area of the hole, after insertion of the adaptor, may function as a vent.

14. A shell according to claim 11, having protrusions at the hole for engagement with the at least second flange for prevention of rotational movement of the adaptor in the hole.

15. A shell according to claim 11, further comprising an end plate containing the hole.

16. A tool for insertion of an adaptor according to claim 1 with a sound tube into a shell hole, the tool having a first protrusion that is adapted for insertion into an output opening of the adaptor, and a second protrusion wherein the space between the first and second protrusions is adapted to accommodate a wall of the adaptor.

17. A tool according to claim 16, further having a center hole for checking that the adaptor may be used with a particular shell, the center hole having a diameter so that the cross-section of the end of the particular shell with the hole can cover the center hole when the shell has a sufficient size for accommodating an adaptor.

18. A tool according to claim 16, wherein an end of the tool is dimensioned so that the end can enter the shell hole when the shell has a sufficient size for accommodating an adaptor.

19. A method of attaching a sound tube to a shell comprising the steps of
attaching one end of the sound tube to an adaptor according to claim 1,
inserting the other end of the sound tube into a hole in the shell from the outside of the shell,
passing the sound tube through the hole, and
mounting the adaptor in the hole whereby the sound tube is attached to the shell.
